(19) United States
(12) Patent Application Publication
(10) Pub. No.: US 2014/0155731 A1
(43) Pub. Date: Jun. 5, 2014

(54) METHOD FOR IDENTIFYING NEUROPROTECTIVE COMPOUNDS AND/OR NEUROREGENERATION STIMULATORS BY FRACTIONAL ANISOTROPY MEASUREMENTS BY DIFFUSION-BASED MRI SCANNING

(71) Applicants: Louis Puybasset, Paris (FR); Damien Galanaud, Paris (FR); Habib Benali, Sainte-Genevieve-des-Bois (FR); Vincent Perlbarg, Paris (FR); Stéphane Lehericy, Paris (FR)

(72) Inventors: Louis Puybasset, Paris (FR); Damien Galanaud, Paris (FR); Habib Benali, Sainte-Genevieve-des-Bois (FR); Vincent Perlbarg, Paris (FR); Stéphane Lehericy, Paris (FR)

(73) Assignee: ASSISTANCE PUBLIQUE - HÔPITAUX DE PARIS, Paris (FR)

(21) Appl. No.: 14/119,226

(22) PCT Filed: May 23, 2012
(86) PCT No.: PCT/FR2012/051164

§ 371 Date: Feb. 20, 2014

(30) Foreign Application Priority Data
May 23, 2011 (FR) 1154469

(51) Int. Cl. A61B 5/00; G01R 33/485; A61B 5/055

(52) U.S. Cl. CPC A61B 5/4848; A61B 5/055; A61B 5/0042; G01R 33/485; A61B 5/7225
USPC 600/410

(57) ABSTRACT

The present invention relates to a method for monitoring the effectiveness of a treatment on neuroprotection and to a method for identifying candidate molecules that are neuroprotectors and/or neuronal growth stimulators. The present invention can be used, in particular, in the field of pharmaceutics, in the field of scientific research and in the field of clinical trials and validation of therapeutic substances.

### TECHNICAL FIELD

The present invention relates to a method for monitoring the effectiveness of a treatment on neuroprotection and to a method for identifying candidate neuroprotective compounds and/or compounds stimulating neural growth.

The present invention is notably applicable in the pharmaceutical field, in the field of scientific research and in the field of clinical trials and validation of therapeutic substances.

In the following description, the references in square brackets ([ ]) refer to the list of references given at the end of the text, where of all of said references and all of their contents are herein incorporated by reference in their entirety.

### BACKGROUND

At the present time, it is extremely difficult to prove the effectiveness of medicines, for example neuroprotectors or neurostimulants, for example on a simple clinical evaluation at one or two years after a cranial trauma, a cerebral vascular accident, an aneurismal meningeal hemorrhage, an intra-cerebral hematoma, a cerebral anoxia of circulatory origin or any other etiology of acute neurological lesions.

In recent years, following favorable results obtained among animals, some fifteen clinical trials have evaluated the neuroprotective effects of a number of molecules such as glutamatergic antagonists, calcic antagonists, anti-oxidants, synthetic cannabinoids, bradykinin inhibitors, etc. Unfortunately, none of these studies has been able to demonstrate a benefit in terms of improving the clinical prognosis of patients with cranial trauma, or patients who have suffered a cerebral vascular accident, an aneurismal meningeal hemorrhage, an intra-cerebral hematoma, a cerebral anoxia of circulatory origin or any other etiology of acute neurological lesions.

A number of explanations can be given to explain these difficulties:

1. The medicines assessed have effectively no effectiveness, which could, for example, be the case with synthetic cannabinoids.
2. There was some effectiveness, but only in a sub-group. In fact, cranial trauma is a syndrome and not a sickness and the physiopathological mechanisms involved in contusions are probably very different from those observed in diffuse axonal lesions.

There is therefore a real need to find a procedure and/or an objective method that makes it possible to effectively evaluate the effect of a molecule on, for example, cranial trauma, for example according to the lesion-causing mechanism involved.

3. There was some effectiveness, but weak, not detectable on clinical categorization of the neurological prognosis produced with a scale such as that of GOSE, too dependent on other factors of environmental type.

These effects would have been visible if the study had included more subjects (lack of power).

There is therefore a real need to find a procedure and/or a method that makes it possible to evaluate, with a limited number of subjects, the effects of molecules for neuroprotection and/or neurostimulation.

4. There was some effectiveness, but weak, very much lower as an absolute value than the effects of the astonishing factors. Such is, for example, the case of statins in aneurismal meningeal hemorrhage; they are effective on a biomarker, S100, but their effect is “drowned” by the influence of the complications of the endovascular or operational procedure and of the clinical grade. In all, the deleterious effects on S100, of the complications and of the clinical grade far outweigh the positive effect of the statins which become clinically invisible.

There is therefore a real need to find a procedure and/or a method that mitigates these defects, drawbacks and obstacles of the prior art, in particular a process that makes it possible notably to control the number of subjects, to obtain reproducible and normalized results, to reduce the costs and improve the evaluation of molecules likely to have a neuroprotective and/or neural growth stimulant effect.

### BRIEF SUMMARY

The present invention specifically makes it possible to resolve and overcome the abovementioned obstacles and drawbacks of the prior art by providing a method for monitoring the effectiveness of a treatment on neuroprotection comprising:
a) measurement of the fractional anisotropy FA₁ on an image obtained by Magnetic Resonance Imaging (MRI) of the brain of a patient before said treatment,
b) measurement of the fractional anisotropy FA₂ on an MRI imaging of the brain of said patient after said treatment,
c) comparison of the fractional anisotropy values and calculation of a score (S) by using the following formula:

S=FA₂/FA₁,

a value of S greater than or equal to 1 indicating that the treatment is a neuroprotective and/or neural growth stimulator treatment.

In particular, the subject of the present invention is a method for monitoring the effectiveness of a treatment on neuroprotection comprising:
a) measurement of the fractional anisotropy FA₁ in at least one region of interest of the brain on an image obtained by Magnetic Resonance Imaging (MRI) of the brain of a patient before said treatment,
b) measurement of the fractional anisotropy FA₂ of said at least one region of interest of the brain on an MRI imaging of the brain of said patient after said treatment,
c) comparison of the fractional anisotropy values and calculation of a score (S) for each region of interest by using the following formula:

S=FA₂/FA₁,

a value of S greater than or equal to 1.08 indicating that the treatment is a neuroprotective and/or neural growth stimulator treatment.

In the present a value of S greater than 1.08 should be understood to mean a value of S greater than 1 plus 2 standard deviations of the fluctuation observed during the same period of time in the same region of interest in the test subjects in at least one of the defined regions of interest. In practice, on average, the fluctuation of the FA observed spontaneously in healthy volunteers is 0±4% after an average delay of two years. This fluctuation of 4% represents one standard deviation (measurement representing the average measured over all the regions of interest in twelve healthy volunteer subjects in two years). According to the invention, a medicine is effective if the fluctuation is greater than two standard deviations of the spontaneous fluctuation, i.e. 8%. According to the invention, a value of S greater than or equal to 1.08 in at least one of the regions of interest studied indicates that the treatment is a neuroprotective and/or neural growth stimulator treatment.

In the present, “MRI” means a medical imaging method based on the magnetic resonance effect, which makes it possible to obtain tomographic images of tissues, for example of soft tissues.

In the present, “MRI image” means any image obtained from an MRI device, for example a 1.5 Tesla, 3.0 Tesla or 7.0 Tesla MRI apparatus, for example from the company Philips, from the company General Electric (GE), or from the company Siemens.

According to the invention, the MRI image can be any image obtained by an MRI device, for example a non-weighted image, preferably a diffusion-weighted image.

In the present “diffusion MRI” means a sequence sensitive to the local diffusion characteristics of the water molecules in the tissues as described in Basser et al. 1994 [1]. In the brain, the organization of the axons in fiber bundles induces an anisotropic diffusion of the water molecules, more significant in the direction of the fibers than in the transversal plane. The MRI of the diffusion tensor (DTI) makes it possible to quantify this anisotropy locally by measuring the local diffusion in the three main directions (λ1, λ2 and λ3) of the model of the tensor based on diffusion measurements repeated in different directions of space as described in Basser and Pierpaoli 1996 [2]. These measurements make it possible to evaluate the axial diffusivity or AD (AD=λ1), the radial diffusivity or RD (RD=(λ2+λ3)/2), the mean diffusivity or MD (MD=(λ1+λ2+λ3)/3) and the fractional anisotropy or FA (FA=sqrt(½) sqrt((λ1−λ2)²+(λ1−λ3)²+(λ2−λ3)²)/sqrt(λ1²+λ2²+λ3²)). In the context of cranial trauma, a local lowering of FA is interpreted as a loss of integrity of the white matter fibers due to the presence of lesions. The lowering of FA is associated with an increase in RD linked to a local loss of myelin and to a lowering of AD linked to axonal lesions.

In the present, “measurement of fractional anisotropy” means, for example, the measurement described in Basser and Pierpaoli 1996 [2] calculated from the three first specific values of the model of the tensor (λ1, λ2 and λ3) such that:

FA=sqrt(½)*sqrt((λ1−λ2)²+(λ1−λ3)²+(λ2−λ3)²)/sqrt (λ1²+λ2²+λ3²)

In the present, “patient” means any individual likely to have, for example, a cerebral lesion, for example an acute cerebral lesion, possibly, for example, a mammal, preferably a human.

In the present, the patient can be a patient that has suffered, for example, a cerebral lesion and/or cranial trauma and/or a meningeal hemorrhage, for example an aneurismal meningeal hemorrhage and/or an ischemic accident and/or a hemorrhagic accident, for example an intraparenchymal hemorrhagic accident and/or cerebral anoxia, for example following a cardiac or circulatory arrest.

It may also be a patient affected by a chronic disease of the white matter, for example a patient affected by end plate sclerosis.

In the present, “treatment” means, for example, a medical treatment, for example allopathic, involving the taking of molecules, for example chemical molecules, for example molecules obtained by organic synthesis, molecules of biological origins, for example proteins, molecules originating from living organisms, for example mammals, microorganisms, plants and/or synthesized by living organisms, for example proteins, nucleic acid molecules, or any other non-chemical treatment, for example re-education, or any other treatment based on cell therapy, for example the injection of stem cells, the injection of dedifferentiated nerve cells.

In the present, the measurements of fractional anisotropy in steps a) and b) can be performed in identical or different regions of the brain, preferably in identical regions.

In the present, the measurements of fractional anisotropy of steps a) and b) can be performed in at least one of the regions of the brain, also called region of interest, chosen from the middle cerebellar peduncle (ICBM #1), the anterior brain stem (ICBM #2,7,8), the posterior brain stem (ICBM #9,10,11,12,13,14), the genu of the corpus callosum (ICBM #3), the body of the corpus callosum (ICBM #4), the splenium of the corpus callosum (ICBM #5), the right cerebral peduncle (ICBM #15), the left cerebral peduncle (ICBM #16), the right sagittal stratum (ICBM #21,29,31,47), the left sagittal stratum (ICBM #22,30,32,48), the right superior longitudinal fascicle (ICBM #41), the left superior longitudinal fascicle (ICBM #42), the anterior limb of the right internal capsule (ICBM #17), the anterior limb of the left internal capsule (ICBM #18), the posterior limb of the right internal capsule (ICBM #19), the posterior limb of the left internal capsule (ICBM #20), the right external capsule (ICBM #33), the left external capsule (ICBM #34), the right radiate crown (ICBM #23,25,27), the left radiate crown (ICBM #24,26,28).

In the present document, “ICBM #n” refers to the nth region of the atlas of 48 regions of white matter constructed from diffusion data from 81 healthy subjects (the “ICBM-DTI-81” atlas (Mori et al. 2005 [9]) available in the FSL software (Smith et al. 2004 [7]).

In the present, the measurements of fractional anisotropy can be performed, for example, in at least one of the regions of the skeleton of the white matter fascicles defined from the TBSS (Tract-Based Spatial Statistics) approach as described in Smith et al. 2006 [8]. It can be, for example, at least one region, also called region of interest, of the brain chosen from the middle cerebellar peduncle (ICBM #1), the anterior brain stem (ICBM #2,7,8), the posterior brain stem (ICBM #9,10,11,12,13,14), the genu of the corpus callosum (ICBM #3), the body of the corpus callosum (ICBM #4), the splenium of the corpus callosum (ICBM #5), the right cerebral peduncle (ICBM #15), the left cerebral peduncle (ICBM #16), the right sagittal stratum (ICBM #21,29,31,47), the left sagittal stratum (ICBM #22,30,32,48), the right superior longitudinal fascicle (ICBM #41), the left superior longitudinal fascicle (ICBM #42), the anterior limb of the right internal capsule (ICBM #17), the anterior limb of the left internal capsule (ICBM #18), the posterior limb of the right internal capsule (ICBM #19), the posterior limb of the left internal capsule (ICBM #20), the right external capsule (ICBM #33), the left external capsule (ICBM #34), the right radiate crown (ICBM #23,25,27), the left radiate crown (ICBM #24,26,28).

Preferably, according to the invention, the measurements are performed in at least 2, or 3, or 4, or 5, or 6, or 7, or 8, or 9, or 10, or 11, or 12, or 13, or 14, or 15, or 16, or 17, or 18, or 19, or 20 of the regions of the brain, also called regions of interest, chosen from the middle cerebellar peduncle (ICBM #1), the anterior brain stem (ICBM #2,7,8), the posterior brain stem (ICBM #9,10,11,12,13,14), the genu of the corpus callosum (ICBM #3), the body of the corpus callosum (ICBM #4), the splenium of the corpus callosum (ICBM #5), the right cerebral peduncle (ICBM #15), the left cerebral peduncle (ICBM #16), the right sagittal stratum (ICBM #21,29,31,47), the left sagittal stratum (ICBM #22,30,32,48), the right superior longitudinal fascicle (ICBM #41), the left superior longitudinal fascicle (ICBM #42), the anterior limb of the right internal capsule (ICBM #17), the anterior limb of the left internal capsule (ICBM #18), the posterior limb of the right internal capsule (ICBM #19), the posterior limb of the left internal capsule (ICBM #20), the right external capsule (ICBM #33), the left external capsule (ICBM #34), the right radiate crown (ICBM #23,25,27), the left radiate crown (ICBM #24,26,28).

Preferably, the measurements are performed in a plurality or all of the following regions of the brain, also called regions of interest: the middle cerebellar peduncle (ICBM #1), the anterior brain stem (ICBM #2,7,8), the posterior brain stem (ICBM #9,10,11,12,13,14), the genu of the corpus callosum (ICBM #3), the body of the corpus callosum (ICBM #4), the splenium of the corpus callosum (ICBM #5), the right cerebral peduncle (ICBM #15), the left cerebral peduncle (ICBM #16), the right sagittal stratum (ICBM #21,29,31,47), the left sagittal stratum (ICBM #22,30,32,48), the right superior longitudinal fascicle (ICBM #41), the left superior longitudinal fascicle (ICBM #42), the anterior limb of the right internal capsule (ICBM #17), the anterior limb of the left internal capsule (ICBM #18), the posterior limb of the right internal capsule (ICBM #19), the posterior limb of the left internal capsule (ICBM #20), the right external capsule (ICBM #33), the left external capsule (ICBM #34), the right radiate crown (ICBM #23,25,27), the left radiate crown (ICBM #24,26,28).

According to the invention, the value of the fractional anisotropy FA₁ and/or FA₂ can be equal to the average of the measured FAs or to the measurement of the FA measured in each region.

In other words, the value of the fractional anisotropy FA₁ and/or FA₂ can be equal to the average of the fractional anisotropies measured independently in each region of interest.

Thus, if the measurement of the fractional anisotropy is performed in a region, the FA value will be equal to the average of the values measured, for example, for each voxel of the image obtained by MRI of the region.

If the value of the fractional anisotropy is measured in several regions of interest, each region will have a specific FA value corresponding to the average of the values measured for each voxel of the image obtained by MRI of each region.

In this embodiment, the measurement of S will be able to be calculated independently for each region of interest.

In the present, the measurement of the fractional anisotropy in step a) can be performed on an MRI image taken in a period of from 1 to 180 days, for example following a cerebral lesion, from 48 hours to 31 days, within a delay less than 31 days.

In the present, the measurement of the fractional anisotropy in step b) can be performed on an MRI image taken in a period of approximately 1 to several months, for example 1 to 12 months, for example 1 to 9 months, for example 6 months, 1 to 6 months, for example 3 months, 1 to 3 months, approximately 1 year to several years, for example 1 to 5 years, 1 to 3 years, 1 to 2 years following the measurement of fractional anisotropy in step a). The measurement of the fractional anisotropy in step a) is the first measurement of fractional anisotropy.

The method of the invention may also comprise a step d) of measuring the axial diffusivity, radial diffusivity, mean diffusivity, and apparent diffusion coefficient.

In the present, “axial diffusivity” means the first specific value λ1 of the model of the tensor calculated from diffusion-weighted MRI images and corresponding to the main direction of diffusion.

In the present, “radial diffusivity” means the average of the second and third specific values (λ2+λ3)/2 of the model of the tensor calculated from diffusion-weighted MRI images and corresponding to the main direction of diffusion.

In the present, “mean diffusivity” means the average of the three specific values (λ1+λ2+λ3)/3 of the model of the tensor calculated from diffusion-weighted MRI images and corresponding to the main direction of diffusion.

In the present, “apparent diffusion coefficient” means the measurement of the mobility of the water molecules locally by comparison of the MRI images without diffusion weighting (b=0) and those which are diffusion-weighted (b>0).

The method of the present invention is advantageously applicable in the medical field where it will be able to be used, for example, in clinical trials in order to determine and/or validate the effectiveness of a treatment on neuroprotection.

Furthermore, the method of the invention advantageously makes it possible to obtain a result that is reliable and reproducible, and that can be compared.

The subject of the present invention is also a method for identifying a neuroprotective and/or neural growth stimulating candidate molecule comprising:
a) measurement of the fractional anisotropy FA₁ on an image obtained by Magnetic Resonance Imaging (MRI) of the brain of a patient before treatment with said compound,
b) measurement of the fractional anisotropy FA₂ on an MRI imaging of the brain of said patient after said treatment,
c) comparison of the fractional anisotropy values and calculation of a score (S) by using the following formula:

S=FA₂−FA₁,

a value of S greater than or equal to 1 indicating that the molecule is a neuroprotector and/or stimulates neural growth.

In particular, the subject of the present invention is also a method for identifying a neuroprotective and/or neural growth stimulating candidate molecule comprising:
a) measurement of the fractional anisotropy FA₁ in regions of interest of the brain on an image obtained by Magnetic Resonance Imaging (MRI) of the brain of a patient before said treatment,
b) measurement of the fractional anisotropy FA₂ in the same regions of interest of the brain on an MRI imaging of the brain of said patient after said treatment,
c) comparison of the fractional anisotropy values and calculation of a score (S) for each region of interest by using the following formula:

S=FA₂/FA₁,

a value of S greater than 1.08 indicating that the molecule is neuroprotective and/or stimulates neural growth.

In the present, neuroprotective means conserving the neural structure and/or reducing neurodegeneration, for example reducing and/or totally stopping neurodegeneration. In the present, the reduction and/or total stoppage of neuroregeneration can be evaluated, for example, by analyzing changes in radial and axial diffusivities.

In the present, neural growth stimulator means, for example, an increase of neural growth; it may be, for example, an increase, for example in the value of the measurement of the fractional anisotropy of at least 8% in a patient after treatment.

In particular, it may be an increase of at least two standard deviations of the value of the measurement of fractional anisotropy, that is to say at least two times the measurement fluctuation observed during the same period of time in the same region of interest in the control subjects.

In the present, “candidate molecule” means any molecule that is to be tested. It may be, for example, chemical and/or biological molecules. It may concern, for example, therapeutic molecules that can be used for the treatment of pathologies, medicines, for example any substance or compound known to those skilled in the art and having curative and/or preventive properties with respect to pathologies, lesions, trauma, human or animal sicknesses. It may be, for example, a pharmaceutical product for human and/or veterinary use.

Also the subject of the present invention is the candidate molecule as neuroprotector and/or neural growth stimulant identified by the method of the invention.

Also the subject of the present invention is the candidate compound identified for its use as neuroprotective and/or neural growth stimulating medicine.

The present invention therefore advantageously makes it possible to evaluate the effectiveness of molecules as neuroprotector by providing a reliable, reproducible and comparable result. Furthermore, the method of the invention advantageously makes it possible to validate the effectiveness of compounds following a clinical trial.

The present invention also makes it possible to identify new candidate molecules likely to have a neuroprotective and/or neurostimulative action that can, for example, be used in neural pathologies, for example Alzheimer's disease and/or be used following a cerebral lesion in order, for example, to retain the integrity of the nerve cortex and reduce neural degeneracy, in particular of the white matter.

Furthermore, the present invention makes it possible to compare the effectiveness of molecules relative to one another, for example relative to molecules already known for the abovementioned applications.

Other advantages may also become apparent to those skilled in the art on reading the examples below, illustrated by the appended figures, given by way of illustration.

### BRIEF DESCRIPTION OF THE FIGURES

FIG. 1 is an image representing the skeleton of the main FA fascicles overlaid on the average FA image over 58 healthy volunteers FMRID58_FA.

FIG. 2 is an image representing the mask of the regions of interest for extraction of the FA.

FIG. 3 is a diagram representing the average values of the regional FAs normalized for the two groups of patients, solid squares: good prognoses, solid rhomboids: poor prognosis. For each region, the normal value is 1. The regions are ordered by ascending values of the good prognosis group. The X axis indicates the different regions of the brain, the Y axis the average FA values.

FIG. 4 is a diagram representing the FA differences measured and the X axis the regions of the brain in which these values have been measured.

### EXAMPLES

### Example 1

### Determination of the Regions for the Measurement of the Fractional Anisotropy (FA), Method for Extracting MRI Biomarkers

a) MRI Acquisitions of the Diffusion Tensor

A DTI acquisition comprises a weighted acquisition at T2 (corresponding to a factor b=0) and acquisitions with diffusion weighting gradients (b˜=1000 s/mm²). To apply the model of the tensor, acquisition with gradients in at least six different directions of space was necessary.

b) Image Preprocessing Operations

A series of raw DTI data preprocessing operations was carried out using the FSL software (http://www.fmrib.ox.ac.uk/fsl/, Smith et al. 2004 [7]:
correction of the distortions induced by the Foucault currents (with the “eddycorrect” function). This correction comprised realigning (rigid realignment) the diffusion-weighted volumes on the T2-weighted volume as described in Jenkinson et al. 2002 [4].
Extraction of the mask of the brain by removing from the volume all the non-brain tissues (with the bet function) as described in Smith 2002 [6].
Calculation of the three specific values (λ1, λ2 and λ3) of the model of the tensor for each voxel as described in Basser et al. 1996[2], making it possible to calculate the parametric map of fractional anisotropy (FA) (with the dtifit function).

In particular, the regional diffusion measurements were obtained as follows. The calculations were performed on a workstation equipped with a Linux operating system (Ubuntu 10.04 LTS).

The brain MRI acquisition was performed by diffusion tensor imaging comprising a T2 weighted volume (corresponding to a factor b=0) and a series of volumes (at least 6) which are diffusion-weighted (corresponding to a factor b>0, typically b varies between 500 and 1500 s/mm²). The series of diffusion-weighted images was saved in the DICOM (Digital Imaging and Communications in Medicine) format (http://medical.nema.org/) and exported to an independent workstation.

The DICOM (Digital Imaging and Communications in Medicine) images obtained were in 4D volume in NIFTI-1 format (Cox et al. 2004 [12]) (nifti.nimh.nih.gov/nifti-1) using the dcm-2nii software (Rorden & Brett, 2000 [13]) http:www.mccauslandcenter.sc.edu/mricro/mricron/dcm2nii.html or the MRIconvert software as follows:

The DICOM files to be converted were in the folder ˜/DTI_DICOM and a terminal was opened, and the following command was launched:

dcm2nii ˜/DTI_DICOM
the following files were then written into this same folder: the file from the diffusion 4D volume named dti4D.nii, the file named dti4D.bval describing the value of the factor b for each gradient direction and the file named dti4D.bvec describing the coordinates of the different gradient directions. These three files were moved into a folder whose access path is ˜/DTI_NII.

The calculation of the parametric maps of fractional anisotropy (FA), of radial diffusivity (RD), of axial diffusivity (AD) and of mean diffusivity (MD) was performed as follows:

This calculation was performed by a sequence of procedures implemented in the FSL software (version 4.1) (Smith et al. 2004 [14]) available free at (http://www.fmrib.ox.ac.uk/fsl/).
correction of the distortions induced by the eddy currents

This correction comprises realigning (rigid realignment) the diffusion-weighted volumes on the T2 weighted volume as described in Jenkinson et al. 2002 [4], the following command was launched:
eddy_correct˜/DTI_NII/dti4D.nii˜/DTI_NII/corr_dti4 D 0
the corrected 4D volume is ˜/DTI_NII/corr_dti4D.nii.
extraction of the mask of the brain

This step comprises removing from the volume all the non-brain tissues as described in Smith 2002[6]. The following command was launched:
bet2 ˜/DTI_NII/corr_dti4D.nii ˜/DTI_NII/brain_corr_dti4D -f 0.2 -m

The file corresponding to the masked 4D volume is ˜/DTI_NII/brain_corr_dti4D.nii.

The file corresponding to the binary brain mask is ˜/DTI_NII/brain_corr_dti4D_mask.nii.
Calculation of the volumes of FA, MD, AD and RD

The aim is to calculate the three specific values (λ1, λ2 and λ3) of the model of the tensor for each voxel as described in Basser et al. 1996 [2] and combine them to calculate the four parameters of interest: axial diffusivity or AD (AD=λ1), radial diffusivity or RD (RD=(λ2+λ3)/2), mean diffusivity or MD (MD=(λ1+λ2+λ3)/3) and fractional anisotropy or FA (FA=sqrt(½)*sqrt((λ1−λ2)²+(λ1−λ3)²+(λ2−λ3)²)/sqrt(λ1²+λ2²+λ3²)). For this, the following command was launched:
dtifit -data=˜/DTI_NII/brain_corr_dti4D.nii --out=˜/DTI_NII/dti_corr_dti4D --mask=˜/DTI_NII/brain_corr_dti4D_mask --bvecs=˜/DTI_NII/dti4D.bvec --bvals=˜/DTI_NII/dti4D.bval

The file corresponding to the FA volume is ˜/DTI_NII/dti_corr_dti4D_FA.nii, the file corresponding to the MD volume is ˜/DTI_NII/dti_corr_dti4D_MD.nii, the file corresponding to the AD volume is ˜/DTI_NII/dti_corr_dti4D_L1.nii and the file corresponding to the RD volume is ˜/DTI_NII/dti_corr_dti4D_Lt.nii.

At the end of this preprocessing step, each patient was characterized by an image representing an FA map.

c) Extraction of the Regional Parameters

So as to compare the maps with one another, the FA maps were projected into a standard space. For this, the individual FA maps were first of all realigned by a non-linear realignment FNIRT (FMRIB's Non-Linear Image Registration tool) [Andersson et al. 2007a, 2007b] in a reference space characterized by a reference image calculated on 58 healthy subjects (FRMRIB58_FA). So as to take account only of the maximum FA values along the fascicles, these maximum local values were projected onto the skeleton of the main FA fascicles (see FIG. 1) according to the TBSS method described in Smith et al. 2006 [8].

FIG. 1 is an image representing the skeleton of the main FA fascicles superimposed on the average FA image over 58 healthy volunteers. As represented in this figure, it can clearly be seen that this skeleton represents the centers common to the group of the main white matter fascicles in the brain.

In particular, the parametric volumes were normalized spatially as follows:

The FA volume was projected into a standard space to allow for the extraction of the regional parameters according to the spatial reference of the atlas used to define the regions of interest. For this, a four-step “tract-based spatial statistics” (TBSS) method described in Smith et al. 2006 [8] was used. A “TBSS” folder located at ˜/DTI_NII/tbss was created, into which the file corresponding to the FA volume dti_corr_dti4D_FA.nii was copied. Before launching the procedures, it was essential to go to the folder :cd ˜/DTI_NII/tbss

### TBSS-1: Preprocessing

This step lightly erodes the images and eliminates the first and last cuts from the volume. For this, the following command was launched: tbss_{—}1_preproc *

### TBSS-2: Non-Linear Realignment—Calculation of the Transformation

The FA volume was realigned by an FNIRT (FMRIB's Non-Linear Image Registration Tool) non-linear realignment as described in Andersson et al. 2007a [10], 2007b [11] in a reference space characterized by a reference image calculated on 58 healthy subjects (FMRIB58_FA). For this, the following command was launched: tbss_{—}2_reg-T.

### TBSS-3: Non-Linear Realignment—Application of the Transformation

The transformation previously calculated was applied to the FA volume. The volume was projected into the MNI152 1×1×1 mm space. For this, the following command was launched:

tbss_{—}3_postreg - T

The file corresponding to the resulting volume was ˜/DTI_NII/tbss/stats/all_FA.nii.

TBSS-4: Projection of the Values onto the FA Skeleton.

So as to take account only of the maximum FA values along the fascicles, these maximum local values were projected onto the skeleton of the main FA fascicles. For this, a map of the distances to the reference FA skeleton was calculated before performing the projection of the values as described in Smith et al. 2006 [8]). For this, the following command was launched: tbss_{—}4_prestats 0.2

The file corresponding to the volume of the FA values on the skeleton was ˜/DTI_NII/tbss/stats/all_FA_skeletonised.nii.

Application of the transformation to the AD, RD and MD volumes.

The non-linear realignment and the projection onto the FA skeleton were applied in the same way to the AD, RD and MD volumes. For example, for MD, a folder with the access path ˜/DTI_NII/tbss/MD was created into which the file corresponding to the MD volume dti_corr_dti4D_MD.nii was copied and renamed dti_corr_dti4D_FA.nii. From the dossier with the access path ˜/DTI_NII/tbss, the following command was launched :tbss_non_FA MD.

The file corresponding to the volume of the MD values on the skeleton is ˜/DTI_NII/tbss/stats/all_MD_skeletonised.nii.

The file corresponding to the volumes ˜/DTI_NII/tbss/stats/all_L1_skeletonised.nii and ˜/DTI_NII/tbss/stats/all_L1_skeletonised.nii were obtained in the same way.

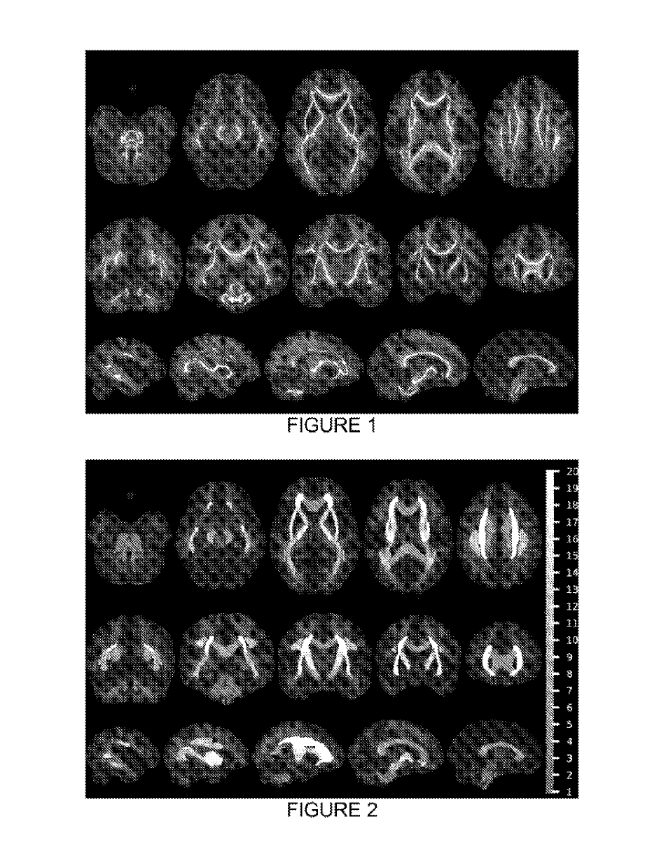

Moreover, 20 regions of interest (ROIs) were defined on the basis of the atlas of 48 regions of white matter constructed from diffusion data from 81 healthy subjects (the atlas “ICBM-DTI-81” available in the FSl software). These 20 ROIs were chosen by a group of experts (two neuroradiologists and one neuro-reaniminator) by taking into account their size (the small original ROIs were eliminated or merged) and their diagnostic interest potential. These 20 regions of interest are represented in FIG. 2, they are indicated by a number from 1 to 20 according to the coloring of the image in correlation with the scale of degradation. It concerns the middle cerebellar peduncle indicated 1 (ICBM #1), the anterior brain stem indicated 2 (ICBM #2,7,8), the posterior brain stem indicated 3 (ICBM #9,10,11,12,13,14), the genu of the corpus callosum indicated 4 (ICBM #3), the body of the corpus callosum indicated ((ICBM #4), the splenium of the corpus callosum indicated 6 (ICBM #5), the right cerebral peduncle indicated 7 (ICBM #15), the left cerebral peduncle indicated 8 (ICBM #16), the right sagittal stratum indicated 9 (ICBM #21,29,31,47), the left sagittal stratum indicated 10 (ICBM #22,30,32,48), the right superior longitudinal fascicle indicated 11 (ICBM #41), the left superior longitudinal fascicle indicated 12 (ICBM #42), the anterior limb of the right internal capsule indicated 13 (ICBM #17), the anterior limb of the left internal capsule indicated 14 (ICBM #18), the posterior limb of the right internal capsule indicated 15 (ICBM #19), the posterior limb of the left internal capsule indicated 16 (ICBM #20), the right external capsule indicated 17 (ICBM #33), the left external capsule indicated 18 (ICBM #34), the right radiate crown indicated 19 (ICBM #23,25,27) and the left radiate crown indicated 20 (ICBM #24,26,28) in FIG. 2.

The 20 regional FA parameters of each patient are the averages in each ROI of the FA on the skeleton as obtained in the field with the access path ˜/DTI_NII/tbss/stats/all_FA_skeletonised.nii.

The 20 MD, AD and RD parameters were calculated in the same way.

The inventors have shown, surprisingly, that the use of these ROIs, that is to say the measurement of the FA in these regions allows, on the one hand, for a local evaluation of the lesions and, on the other hand, for a robust comparison between acquisitions and/or subjects.

Each patient was therefore characterized by more than

FA parameters (average of the FA on the skeleton in each ROI) reflecting the regional integrity of the white matter fascicles. These parameters were extracted by masking of the FA maps projected onto the skeleton with the mask of the 20 ROIs. For these parameters to be able to be interpreted in relation to a reference normal level, the FA value measured in each ROI is normalized relative to an average value calculated on a population of healthy subjects, namely 10 individuals, from the same machine and the same MRI acquisition protocols. This normalization also makes it possible to compare the measurements of one MRI machine with another.

Evaluation of the Biomarkers

d) Populations and Acquisitions

41 patients in a coma after cranial trauma were admitted into the neuro-reanimination department at Pitié-Salpêtrière. They were included in the study if they satisfied the following criteria:
1) need for mechanically assisted respiration for neurological reasons,
2) absence of response to simple commands at the time of the signing of the legal consent form by the authorized representative, at least seven days after the accident,
3) absence of response to simple commands not linked to the administration of sedatives,
4) general clinical condition allowing the patient to be transported,
5) cerebral compliance making it possible to maintain the elongate position for MRI acquisition without the development of intra-cranial hypertension.

MRI acquisitions were performed on these patients in the acute phase of the trauma (time 1) that is to say approximately three weeks after the accident. The inclusion of these patients followed the algorithm described in Lescot et al. 2008 [5]. A clinical evaluation of these patients was conducted at least one year after the trauma to determine their GOS (Glasgow Coma Scale) making it possible to classify them into two groups: 21 patients with favorable prognosis (GOS 4-5) and 20 with unfavorable prognosis (GOS 1-3). A second long-term MRI acquisition (>1 year after the accident) was performed on 18 of the 41 patients (time 2). Finally, an MRI acquisition was performed on the same machine on 15 controlled subjects to allow for the diffusion measurements to be normalized.

The details of the MRI acquisitions are as follows. For the acquisition of the patients in the acute phase (time 1), these were under mechanical ventilation and sedation (sufentanil (20-30 lg/h) and propofol (100-200 mg/h)):
MRI machine: GE Signa 1.5T
Diffusion-weighted sequence, 24 directions
TR/TE=8,000/84.9 ms
24 directions; diffusion b value=700 s/mm
cut thickness=5 mm without hole
27 cuts
field of view=32×32 cm²
matrix 256×256
Diffusion-weighted sequence, 11 directions
TR/TE=13,000/85.9 ms
24 directions; diffusion b value=900 s/mm
cut thickness=3 mm without hole
47 cuts
field of view=28×28 cm²
matrix 256×256

Not all the acquisitions could be done on all the patients. Table 1 shows the detail for the 18 patients reviewed in the consolidated phase, for the examination at time 1, only the 24-direction DTI acquisition was performed on all of the 41 patients:

TABLE 1

| | | | | | Delay |
|---|---|---|---|---|---|
| | | | | | time 2- |
| | | | | 24 d | time 1 |
| Patient | 11 d time 1 | 11 d time 2 | 24 d time 1 | time 2 | (days) |
| 1 | X | X | X | X | 612 |
| 2 | X | X | X | X | 376 |
| 3 | — | — | X | X | 1095 |
| 4 | — | X | X | X | 1088 |
| 5 | — | X | X | X | 1445 |
| 6 | X | X | X | X | 457 |
| 7 | — | X | X | X | 574 |
| 8 | — | X | X | X | 1116 |
| 9 | X | X | X | X | 746 |
| 10 | X | X | X | X | 815 |
| 11 | X | X | X | — | 887 |
| 12 | X | X | X | — | 817 |
| 13 | — | X | X | X | 1226 |
| 14 | — | X | X | X | 1074 |
| 15 | — | — | X | X | 1639 |
| 16 | — | X | X | X | 1342 |
| 17 | X | X | X | — | 614 |
| 18 | X | X | X | — | 1017 |

In table 1, the acquisitions made are represented by (X) and not made by (—) for the 18 patients included for the longitudinal monitoring study. The X characters indicate the examinations where one and the same acquisition was done at both acquisition times. The delay in days between the two acquisitions is indicated in the last column.

e) Results

24-Direction DTI Data from the 41 Patients in the Acute Phase

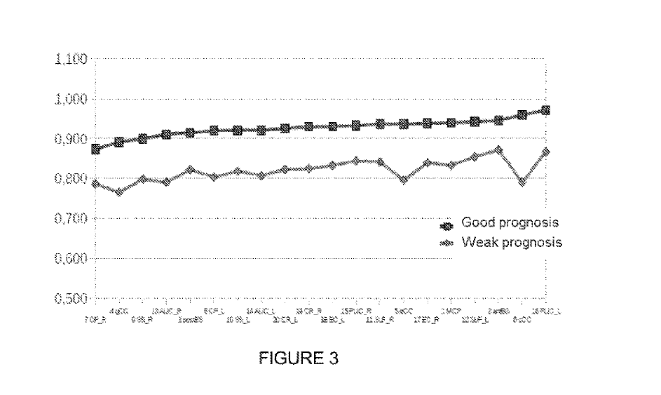

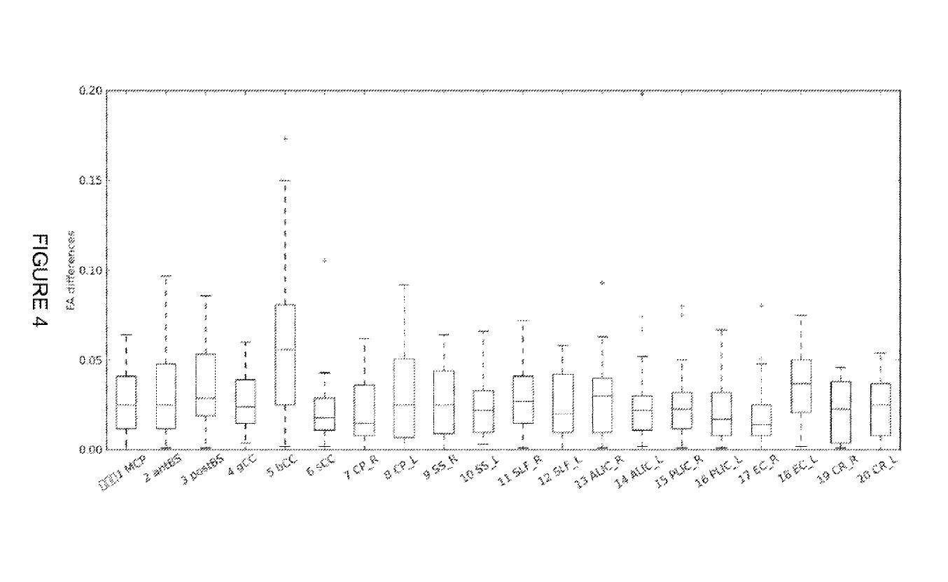

The inventors extracted, on each of these patients, the 20 regional FA values that we have normalized relative to the control values. The average values of these measurements on the two groups (favorable prognosis and unfavorable prognosis) are represented in FIG. 3. As represented in this figure, a lowering of FA was measured: 7% for the patients with good prognosis and 18% for those with poor prognosis. The difference in lowering of FA between the two groups was evaluated statistically by the “two-sample t-test” with p<0.05. This evaluation showed a significant difference for all the regions.

FIG. 3 describes the average values of the normalized regional FAs for the two groups of patients (good and poor prognoses). For each region the normal value is 1. The regions are ordered by ascending values in the good prognosis group.

The 20 regional FA measurements made it possible to quantify the gravity of the white matter lesions; the more severe the lesions in the FA sense, the less good the neurological prognosis for the patient.

11- and 24-Directions DTI Data from the 18 Patients for Longitudinal Monitoring

The FA measurements were calculated in the 20 regions for each patient and each examination (11 and 24 directions, time1 and time2). The distribution of the differences time2−time1 as an absolute value for each patient and each type of acquisition is given in FIG. 4. FIG. 4 represents the different values obtained as a function of the different regions, namely: middle cerebellar peduncle (MCP), anterior brain stem (antBS), posterior brain stem (postBS), genu of the corpus collosium (gCC), body of the corpus callosum (bCC), splenium of the corpus callosum (sCC), right cerebral peduncle (CP_R), left cerebral peduncle (CP_L), right sagittal stratum (SS_R), left sagittal stratum (SS_L), right superior longitudinal fascicle (SLF_R), left superior longitudinal fascicle (SLF_L), anterior limb of the right internal capsule (ALIC_R), anterior limb of the left internal capsule (ALIC_L), posterior limb of the right internal capsule (PLIC_R), posterior limb of the left internal capsule (PLIC_L), right external capsule (EC_R), left external capsule (EC_R), right radiate crown (CR_R) and left radiate crown (CR_L). A paired student test showed that these differences are not significantly different from 0 (p>0.1) regardless of region (results not supplied).

This example therefore demonstrates, in the absence of any specific treatment, that there is no significant modification of the regional FA measurements between an early acquisition and an acquisition in consolidated phase for patients with severe cranial trauma.

The results obtained in this example therefore clearly demonstrate that:
the normalized FA measurements in the 20 chosen regions are relevant biomarkers of the neurological gravity of the lesions after severe cranial trauma.
the normalized FA measurements in the 20 chosen regions remain stable between two remote acquisitions, that is to say at different times, one in the acute phase, the other in the consolidated phase.

In light of these two conclusions, a method comprising the FA measurement makes it possible to measure the effectiveness of medicines, for example of the neuroprotectors administered in the context of acute cerebral pathology.

The average of the FA measurements on the 18 patients (times 1 and 2 together) is m=0.948 and the variance is s=0.006. An increase in this FA measurement of 5% (i.e. d=0.05*m=0.047) with a type I standard error of 5% and a statistical power of 80% (beta=20%) is detected when the number of subjects per group (placebo and treated) is n=15.

Furthermore, as demonstrated in this example, the method of the invention makes it possible to evaluate the effects of different treatments, for example neuroprotective or neural regrowth activator or non-chemical method. It is useful, for example, during clinical trials in phase IIb (validation of the proof of concept). The method of the invention therefore advantageously allows for a drastic reduction of the number of subjects to be included to affirm or deny the effectiveness of a medicine compared to the traditional clinical trials. In this type of approach, a phase III study will be put in place only if an effectiveness on the MRI biomarker has been able to be revealed in phase IIb. The method of the invention therefore makes it possible to test many more molecules at lower cost. It also makes it possible to avoid exposing patients to ineffective treatments and to reduce the number of patients receiving a placebo.

### Example 2

### Evaluation of a Candidate Molecule by Measurement of the Regional FA

Two groups of n patients with severe cranial trauma, one treated, the other with placebo.
Treatment protocol in the acute phase (injection, etc.) or chronic phase.
Imaging protocol to monitor the trend: an early acquisition then acquisition at 3 months, 6 months, and/or one year or even later.
Comparison of the modification over time of the regional FAs between the two groups. Any significant interaction reflects the effectiveness of the treatment, if the treated group has FA values which increase significantly in time relative to the placebo group. The FA measurements made in the placebo group do not vary in time.

### LISTS OF REFERENCES

1. [Basser et al. 1994] P. J. Basser, J. Mattiello, D. LeBihan, MR diffusion tensor spectroscopy and imaging. Biophysical Journal, 66(1):250-267, 1994.
2. [Basser and Pierpaoli 1996] P. J. Basser and C. Pierpaoli, Microstructural and Physiological Features of Tissues Elucidated by Quantitative-Diffusion-Tensor MRI. Journal of Magnetic Resonance, 111(3); 209-219, 1996.
3. [Jenkinson and Smith 2001] M. Jenkinson and S. M. Smith, A global optimization method for robust affine registration of brain images. Medical Image Analysis, 5(2); 143-156, 2001.
4. [Jenkinson et al. 2002] M. Jenkinson and P. R. Bannister, J. M. Brady, and S. M. Smith, Improved optimization for the robust and accurate linear registration and motion correction of brain images. NeuroImage, 17(2):825-841, 2002.
5. [Lescot et al. 2008] T. Lescot, L. Abdennour, A.-L. Boch, L. Puybasset, Treatment of intracranialhypertension. Curr Opin Crit Care, 14:129-134, 2008.
6. [Smith 2002] S. M. Smith, Fast robust automated brain extraction. Human Brain Mapping, 17(3):143-155, 2002.
7. [Smith et al. 2004] S. M. Smith, M. Jenkinson, M. W. Woolrich, C. F. Beckmann, T. E. J. Behrens, H. Johansen-Berg, P. R. Bannister, M. De Luca, I. Drobnjak, D. E. Flitney, R. Niazy, J. Saunders, J. Vickers, Y. Zhang, N. De Stefano, J. M. Brady, and P. M. Matthews, Advances in functional and structural MR image analysis and implementation as FSL. NeuroImage, 23(S1):208-219, 2004.
8. [Smith et al. 2006] S. M. Smith, M. Jenkinson, H. Johansen-Berg, D. Rueckert, T. E. Nichols, C. E. Mackay, K. E. Watkins, O. Ciccarelli, M. Z. Cader, P. M. Matthews, and T. E. J. Behrens, Tract-based spatial statistics: Voxelwise analysis of multi-subject diffusion data. NeuroImage, 31:1487-1505, 2006.
9. [Mori et al. 2005] S. Mori, S. Wakana, L. M. Nagae-Poetscher, and P. C. M. van Zijl. MRI Atlas of Human White Matter. Elsevier, Amsterdam, The Netherlands (2005).
10. [Andersson 2007a] J. L. R. Andersson, M. Jenkinson and S. Smith. Non-linear optimization. FMRIB technical report TR07JA1 from www.fmrib.ox.ac.uk/analysis/techrep
11. [Andersson 2007b] J. L. R. Andersson, M. Jenkinson and S. Smith. Non-linear registration, aka Spatial normalization. FMRIB technical report TR07JA2 from www.fmrib.ox.ac.uk/analysis/techrep
12. [Cox et al. 2004] Robert W. Cox, John Ashburner, Hester Breman, Kate Fissell, Christian Haselgrove, Colin J Holmes, Jack L Lancaster, David E Rex, Stephen M Smith, Jeffrey B Woodward, Stephen C Strother (2004). A (Sort of) New Image Data Format Standard: NifTI-1. NeuroImage, Vol. 22 (2004).
13. [Rorden & Brett, 2000] Rorden, C., Brett, M. (2000). Stereotaxic display of brain lesions. Behavioral Neurology, 12, 191-200.
14. [Smith et al. 2004] S. M. Smith, M. Jenkinson, M. W. Woolrich, C. F. Beckmann, T. E. J. Behrens, H. Johansen-Berg, P. R. Bannister, M. De Luca, I. Drobnjak, D. E. Flitney, R. Niazy, J. Saunders, J. Vickers, Y. Zhang, N. De Stefano, J. M. Brady, and P. M. Matthews (2004). Advances in functional and structural MR image analysis and implementation as FSL. NeuroImage, 23(S1):208-219.

1. A method for monitoring the effectiveness of a treatment on neuroprotection comprising:
a) measurement of the fractional anisotropy FA₁ in at least one region of interest of the brain on an image obtained by Magnetic Resonance Imaging (MRI) of the brain of a patient before said treatment,
b) measurement of the fractional anisotropy FA₂ of said at least one region of interest of the brain on an MRI imaging of the brain of said patient after said treatment,
c) comparison of the fractional anisotropy values and calculation of a score (S) according to the following formula:
S=FA₂/FA₁,
a value of S greater than 1.08 indicating that the treatment is a neuroprotective and/or neural growth stimulator treatment.

2. The method as claimed in claim 1, also comprising a step d) of measuring the axial diffusivity, radial diffusivity, mean diffusivity, and apparent diffusion co efficient.

3. The method as claimed in claim 1, wherein the measurements of fractional anisotropy of steps a) and b) are performed in at least one of the regions of interest of the brain chosen from the middle cerebellar peduncle (ICBM #1), the anterior brain stem (ICBM #2,7,8), the posterior brain stem (ICBM #9,10,11,12,13,14), the genu of the corpus callosum (ICBM #3), the body of the corpus callosum (ICBM #4), the splenium of the corpus callosum (ICBM #5), the right cerebral peduncle (ICBM #15), the left cerebral peduncle (ICBM #16), the right sagittal stratum (ICBM #21,29,31,47), the left sagittal stratum (ICBM #22,30,32,48), the right superior longitudinal fascicle (ICBM #41), the left superior longitudinal fascicle (ICBM #42), the anterior limb of the right internal capsule (ICBM #17), the anterior limb of the left internal capsule (ICBM #18), the posterior limb of the right internal capsule (ICBM #19), the posterior limb of the left internal capsule (ICBM #20), the right external capsule (ICBM #33), the left external capsule (ICBM #34), the right radiate crown (ICBM #23,25,27), the left radiate crown (ICBM #24,26,28).

4. The method as claimed in claim 1, wherein the measurements of fractional anisotropy of steps a) and b) are performed in the regions of interest of the brain chosen from the middle cerebellar peduncle (ICBM #1), the anterior brain stem (ICBM #2,7,8), the posterior brain stem (ICBM #9,10,11,12,13,14), the genu of the corpus callosum (ICBM #3), the body of the corpus callosum (ICBM #4), the splenium of the corpus callosum (ICBM #5), the right cerebral peduncle (ICBM #15), the left cerebral peduncle (ICBM #16), the right sagittal stratum (ICBM #21,29,31,47), the left sagittal stratum (ICBM #22,30,32,48), the right superior longitudinal fascicle (ICBM #41), the left superior longitudinal fascicle (ICBM #42), the anterior limb of the right internal capsule (ICBM #17), the anterior limb of the left internal capsule (ICBM #18), the posterior limb of the right internal capsule (ICBM #19), the posterior limb of the left internal capsule (ICBM #20), the right external capsule (ICBM #33), the left external capsule (ICBM #34), the right radiate crown (ICBM #23,25,27), the left radiate crown (ICBM #24,26,28).

5. The method as claimed in claim 1, wherein the measurement of fractional anisotropy is performed on an MRI image of a patient having suffered a cerebral legion, including cranial trauma, aneurismal meningeal hemorrhagic, ischemic and intraparenchymal hemorrhagic accidents, cerebral anoxia.

6. The method as claimed in claim 5, wherein the measurement of FA₁ is performed on an MRI image taken in a period of 48 hours to 31 days following the brain lesion.

7. The method as claimed in claim 1, wherein the measurement of FA₂ is performed on an MRI image taken in a period of at least one month following the measurement of FA₁.

8. A method for identifying a neuroprotective and/or neural growth stimulative candidate molecule comprising:
a) measurement of the fractional anisotropy FA₁ in at least one region of interest of the brain on an image obtained by Magnetic Resonance Imaging (MRI) of the brain of a patient before said treatment,
b) measurement of the fractional anisotropy FA₂ of said at least one region of interest of the brain on an MRI imaging of the brain of said patient after said treatment,
c) comparison of the fractional anisotropy values and calculation of a score (S) according to the following formula:
S=FA₂/FA₁,
a value of S greater than or equal to 1.08 indicating that the candidate molecule is neuroprotective and/or neural growth stimulative.

9. The method as claimed in claim 8, in which the measurements of fractional anisotropy of steps a) and b) are performed in at least one of the regions of interest of the brain chosen from the middle cerebellar peduncle (ICBM #1), the anterior brain stem (ICBM #2,7,8), the posterior brain stem (ICBM #9,10,11,12,13,14), the genu of the corpus callosum (ICBM #3), the body of the corpus callosum (ICBM #4), the splenium of the corpus callosum (ICBM #5), the right cerebral peduncle (ICBM #15), the left cerebral peduncle (ICBM #16), the right sagittal stratum (ICBM #21,29,31,47), the left sagittal stratum (ICBM #22,30,32,48), the right superior longitudinal fascicle (ICBM #41), the left superior longitudinal fascicle (ICBM #42), the anterior limb of the right internal capsule (ICBM #17), the anterior limb of the left internal capsule (ICBM #18), the posterior limb of the right internal capsule (ICBM #19), the posterior limb of the left internal capsule (ICBM #20), the right external capsule (ICBM #33), the left external capsule (ICBM #34), the right radiate crown (ICBM #23,25,27), the left radiate crown (ICBM #24,26,28).

10. The method as claimed in claim 8, wherein the measurements of fractional anisotropy of the steps a) and b) are performed in the regions of interest of the brain chosen from the middle cerebellar peduncle (ICBM #1), the anterior brain stem (ICBM #2,7,8), the posterior brain stem (ICBM #9,10,11,12,13,14), the genu of the corpus callosum (ICBM #3), the body of the corpus callosum (ICBM #4), the splenium of the corpus callosum (ICBM #5), the right cerebral peduncle (ICBM #15), the left cerebral peduncle (ICBM #16), the right sagittal stratum (ICBM #21,29,31,47), the left sagittal stratum (ICBM #22,30,32,48), the right superior longitudinal fascicle (ICBM #41), the left superior longitudinal fascicle (ICBM #42), the anterior limb of the right internal capsule (ICBM #17), the anterior limb of the left internal capsule (ICBM #18), the posterior limb of the right internal capsule (ICBM #19), the posterior limb of the left internal capsule (ICBM #20), the right external capsule (ICBM #33), the left external capsule (ICBM #34), the right radiate crown (ICBM #23,25,27), the left radiate crown (ICBM #24,26,28).

11. The method as claimed in claim 8, wherein the measurement of fractional anisotropy is performed on an MRI image of a patient having suffered a cerebral lesion, including cranial trauma, aneurismal meningeal hemorrhage, ischemic and intraparenchymal hemorrhagic accidents, cerebral anoxia.

12. The method as claimed in claim 8, wherein the measurement of FA₁ is performed on an MRI image taken in a period of 48 h to 31 days following the cerebral lesion.

13. The method as claimed in claim 8, wherein the measurement of FA₂ is performed on an MRI image taken in a period of at least one month following the measurement of FA₁.
